(19) United States
(12) Patent Application Publication
(10) Pub. No.: US 2005/0187504 A1
(43) Pub. Date: Aug. 25, 2005

(54) Cervical collar utilizing laminated members

(76) Inventor: Michael D. Modglin, Braselton, GA (US)

Correspondence Address: LUEDEKA, NEELY & GRAHAM, P.C., P O BOX 1871, KNOXVILLE, TN, 37901, US

(21) Appl. No.: 10/784,693

(22) Filed: Feb. 23, 2004

(51) Int. Cl. A61F 5/00

(52) U.S. Cl. 602/18

(57) ABSTRACT

A cervical collar having upper and lower posterior laminate bodies each including a low density polyethylene material, a foam material, and a fabric material; and upper and lower anterior laminate bodies each including a low density polyethylene material and a fabric material.

### FIELD OF THE INVENTION

This invention relates generally to cervical collars. More particularly, this invention relates to cervical collars having reduced bulk and improved comfort, fit, and support characteristics.

### BACKGROUND AND SUMMARY OF THE INVENTION

Cervical collars typically have two halves which may be adjustably positioned to encircle a user's neck and secured in place as by straps having mating hook and loop material. Such collars desire improvement in terms of their bulk. Such collars also desire improvement in terms of their comfort, fit and support characteristics.

Accordingly, the invention relates to cervical collars. In a preferred embodiment, the cervical collars according to the invention have upper and lower posterior laminate bodies each including a low density polyethylene material, a foam material, and a fabric material. The collars also include upper and lower anterior laminate bodies each including a low density polyethylene material and a fabric material.

The collars are preferably secured in a tensioned state about the neck of the user, preferably by use of straps, to provide a desired degree of support. The laminate bodies offer reduced bulk as compared to prior collar materials. The laminate bodies also provide collars having improved comfort, fit and support characteristics as compared to prior collars.

### BRIEF DESCRIPTION OF THE DRAWINGS

Further features of preferred embodiments of the invention will become apparent by reference to the detailed description of preferred embodiments when considered in conjunction with the figures, which are not to scale, wherein like reference numbers, indicate like elements through the several views, and wherein,

FIG. 1 is a perspective view showing a cervical collar according to the invention.

FIG. 2 is an exploded perspective view of the collar of FIG. 1.

FIG. 3 is an exterior plan view of a posterior portion of the collar of FIG. 1.

FIG. 4 is a interior plan view of the posterior portion of FIG. 3.

FIG. 5 is a partially exploded interior plan view of the posterior portion of FIG. 3.

FIG. 6 is an exploded exterior view of upper and lower segments of the posterior portion of FIG. 3.

FIG. 7 is an exploded view showing components of the laminate portions of the collar of FIG. 1.

FIG. 8 is an exterior plan view of a anterior portion of the collar of FIG. 1.

FIG. 9 is a interior plan view of the anterior portion of FIG. 8.

FIG. 10 is a partially exploded interior plan view of the posterior portion of FIG. 8.

FIG. 11 is an exploded exterior view of upper and lower segments of the anterior portion of FIG. 8.

FIG. 12 is an exploded interior view of upper and lower segments of the anterior portion of FIG. 8.

FIG. 13 is a perspective view of an adjustably positionable ledge member for use with the collar of the invention.

FIG. 14 is an exploded perspective view showing installation of the ledge member of FIG. 13.

FIG. 15 is an exploded perspective view showing a supplemental strap system for use collars according to the invention.

FIG. 16 is an exploded perspective view of an alternate embodiment of a posterior portion for use with collars according to the invention.

FIG. 17 is an interior plan view of the posterior portion of FIG. 16.

### DETAILED DESCRIPTION

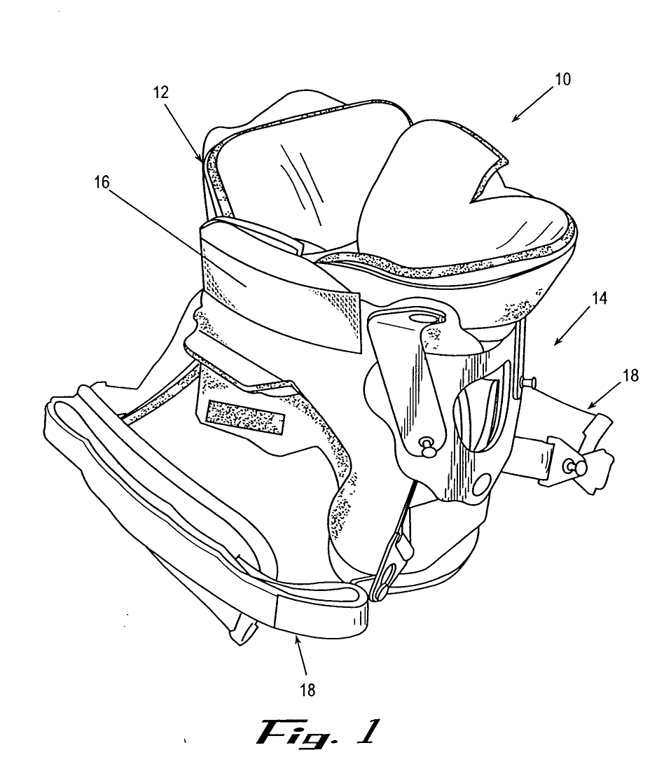

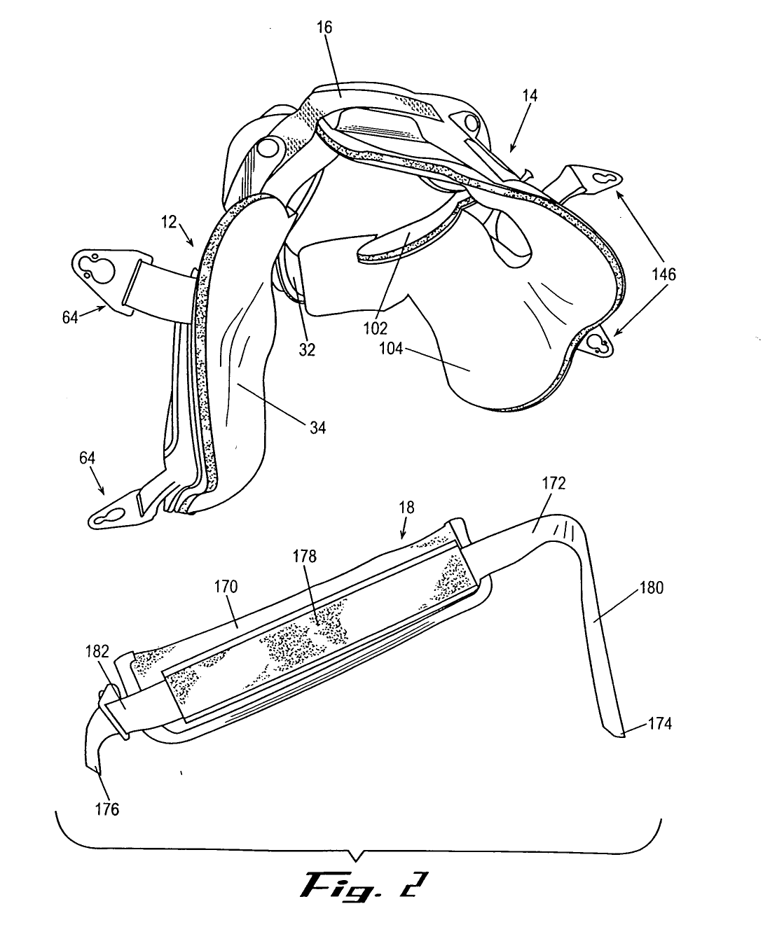

With initial reference to FIGS. 1 and 2, the invention relates to a cervical collar 10 that is lightweight and readily adjustable. The collar 10 includes a posterior portion 12 and an anterior portion 14. The posterior portion 12 is generally configured to conform to the rear of the human neck and the anterior portion 14 is generally configured to conform to the chin and front of the neck.

The posterior and anterior portions 12 and 14 may variably overlap one another when installed around the neck, with the anterior portion 14 preferably overlapping the posterior portion 12. The portions 12 and 14 are preferably secured together as by straps 16. In addition, supplemental strap systems 18 may be provided to extend between portions of the posterior portion 12 and the anterior portion 14. The strap systems 18 are further configured to extend adjacent an under-arm region of the user.

The posterior portion 12 and the anterior portion 14 each utilize bodies made of laminate materials that are substantially flexible in an untensioned state, yet which become sufficiently rigid as to provide the desired support when positioned in an overlapped orientation on the user and tensioned as by the use of the straps 16. The rigidity of the laminate bodies when tensioned also enables the use of complementing rigid members having reduced dimensions, advantageously enabling a more compact and lighter weight construction. The laminate bodies are also configured to be adjustably positionable relative to one another,

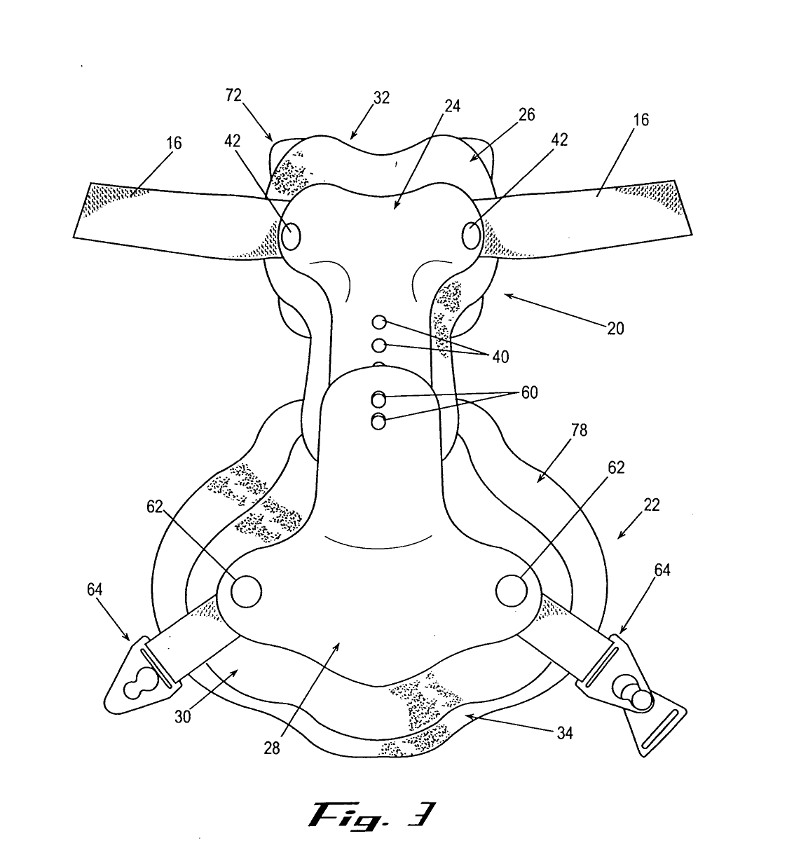

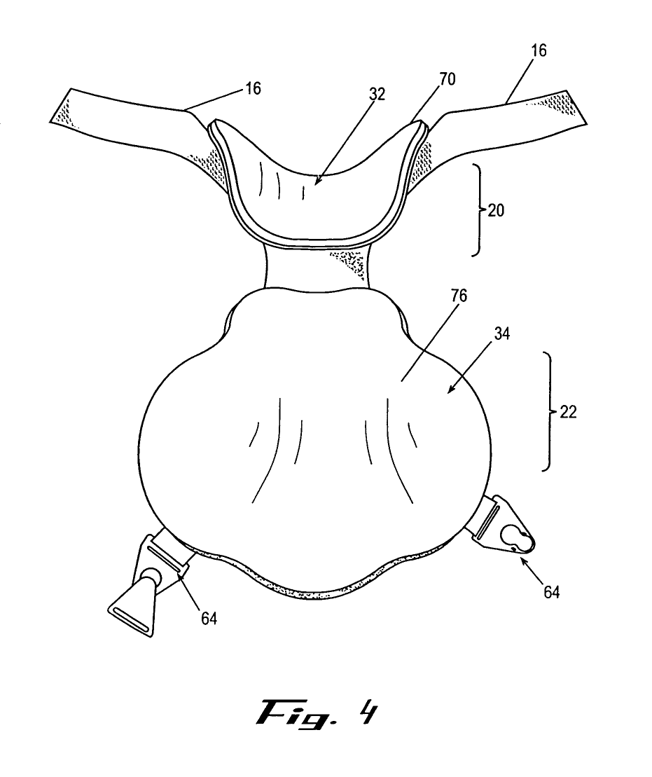

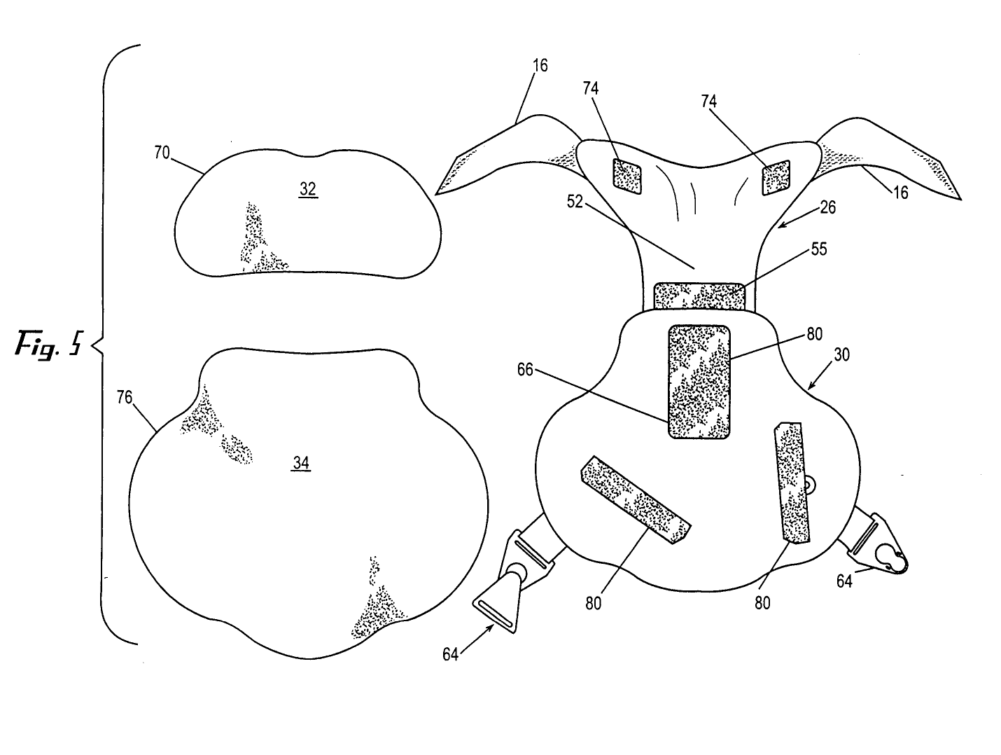

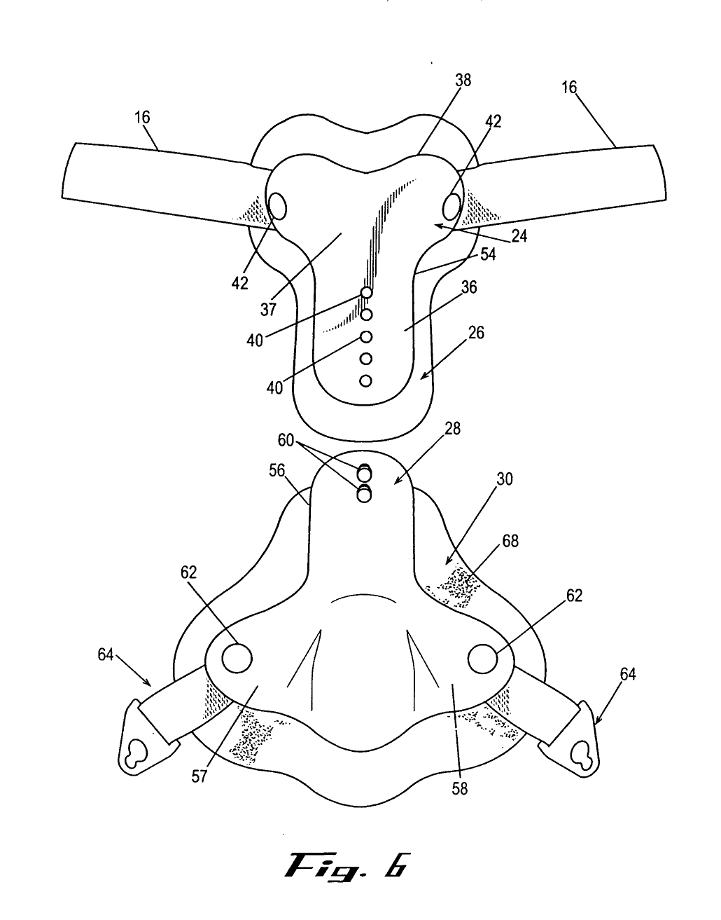

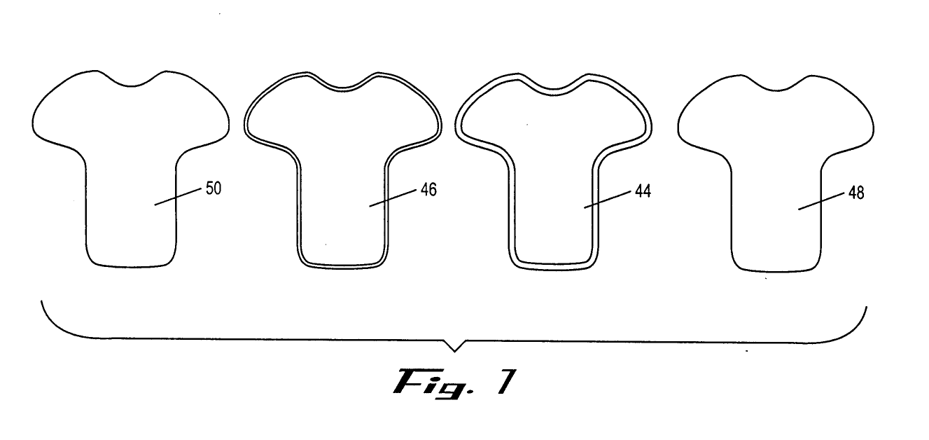

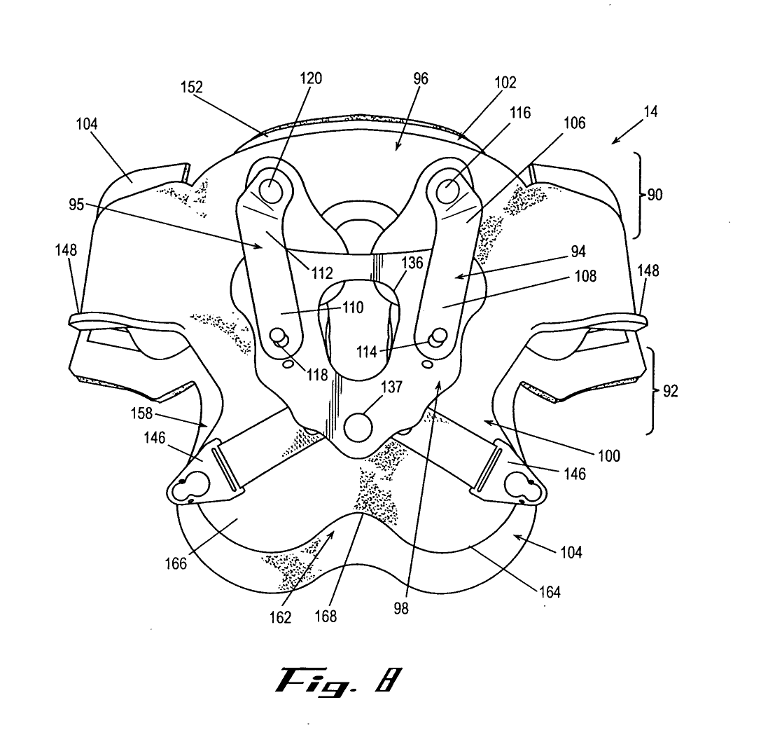

With additional reference now to FIGS. 3-6, the posterior portion 12 preferably includes an upper posterior segment 20 and a lower posterior segment 22 that are adjustably attachable to one another to facilitate fitting of the posterior portion 12 to a user. The upper posterior segment 20 includes an upper posterior rigid member 24 fixedly secured to an upper posterior laminate body 26. The lower posterior segment 22 includes a lower posterior rigid member 28 fixedly secured to a lower posterior laminate body 30.

The upper posterior rigid member 24 is adjustably securable to the lower posterior rigid member 28, and the upper posterior laminate body is 26 is adjustably securable to the lower posterior laminate body 30. An upper interior pad 32 and a lower interior pad 34 (FIG. 5) are preferably releasably attached to interior surfaces of the posterior laminate bodies 26 and 30, respectively, for enhanced comfort.

With additional reference to FIG. 6, the upper posterior rigid member 24 is preferably of one-piece molded plastic construction and is substantially rigid so as to resist deflection under normal use conditions. Preferred plastic materials include ABS plastics and synthetic resinous plastics such as are available under the tradenames KYDEX and BOLTARON. The plastic materials preferably have a thickness of from about 0.090 to about 0.125 inches.

The rigid member 24 is generally contoured to receive upper portions of the neck and spine of the user and has a generally Y-shaped configuration with a leg 36 and a pair of arms 37 and 38. The leg 36 is preferably arched slightly and curved slightly away from the arms 37 and 38 to generally follow the curve of the neck of the user. The arms 37 and 38 are contoured to cradle the neck of the user just below the occipital protuberance region of the skull of the user.

A plurality of apertures 40 extend through the leg 36 for adjusting the position of the upper rigid member 24 relative to the lower rigid member 28, as detailed below. Apertures preferably extend through the ends of the arms 37 and 38 for receiving plastic rivets 42 for attachment of the straps 16 and the upper laminate body 26 to the upper rigid member 24.

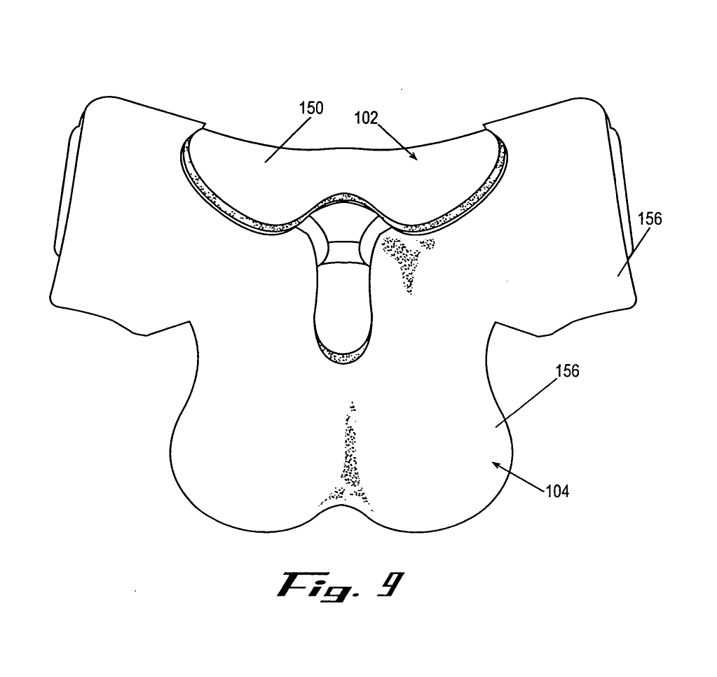

With continued reference to FIG. 6, the upper posterior laminate body 26 is slightly larger than the rigid member 24 and generally Y-shaped to correspond to and overlap the rigid member 24. With reference to FIG. 7, the laminate body 26 preferably includes a generally Y-shaped foam material 44 and a generally Y-shaped polymeric sheet material, preferably a low density polyethylene material 46 sandwiched between generally Y-shaped fabric materials 48 and 50. The body 26 is oriented so that the foam material 44 is adjacent the user.

The foam material 44 is preferably a closed cell foam having a thickness of about ⅛ inch. The low density polyethylene material preferably has a thickness of about 1/16 inch. The fabric materials 48 and 50 are preferably each made of a soft fabric material having exposed surfaces 52 and 54, respectively, that are capable of engaging hook material in the context of mating hook and loop materials, such as that available under the trade name VELCRO. The upper laminate body 26 is preferably made as by vacuum molding with adhesive placed between each layer.

Returning to FIG. 5, a strip of hook material 55, preferably double sided hook material, is attached to the surface 52, and located so as to be matingly engageable with the loop material of the lower laminate body 30 to facilitate adjustable positioning of the upper laminate body 26 relative to the lower laminate body 30.

The lower posterior rigid member 28 is preferably made of the same material as the upper posterior rigid member 24. The rigid member 28 is generally contoured to receive lower portions of the neck and spine of the user and has a generally reverse T-shaped configuration with an upwardly extending extension 56 and a pair of legs 57 and 58. The extension 56 is arched slightly and curves slightly away from the legs 57 and 58 to follow the curve of the neck and the legs 57 and 58 configured to conform to a portion of the upper spine of the user below the neck.

A plurality of fasteners 60 extend through the extension 56 for releasably engaging the apertures 40 of the rigid member 24 for adjusting the position of the upper rigid member 24 relative to the lower rigid member 28. Apertures preferably extend through the ends of the arms 37 and 38 for receiving plastic rivets 62 for attachment of posterior buckle assemblies 64 associated with the strap systems 18 and for attachment of the lower laminate body 30 to the lower rigid member 28.

The lower posterior laminate body 30 is shaped to correspond to and overlap the rigid member 28. The lower posterior laminate body 30 is preferably substantially similar in construction to the laminate body 26 and includes a foam material and a polymeric sheet material, such as a low density polyethylene sheet material, sandwiched between fabric materials having exposed surfaces 66 and 68 that are capable of engaging hook material in the context of mating hook and loop materials, such as VELCRO.

The upper interior pad 32 is preferably made of a soft expanded foam material and includes a soft patient contact surface 70 and an opposite attachment surface 72 capable of engaging hook material in the context of mating hook and loop materials, such as VELCRO. The surface 72 of the pad 32 is releasably engageable with strips of hook material 74 secured to surface 52 of the upper laminate body 26 (FIG. 5). The hook material 74 is preferably double sided so as to releaseably engage the loop material of the surface 52 for securement of the material 74 to the surface 52.

The lower interior pad 34 is preferably made of a soft expanded foam material and includes a soft patient contact surface 76 and an opposite attachment surface 78 capable of engaging hook material in the context of mating hook and loop materials, such as VELCRO. The surface 72 of the pad 32 is releasably engageable with strips of hook material 80 preferably secured to surface 66 of the lower laminate body 30 in the manner of the hook material 74 (FIG. 5).

With additional reference now to FIGS. 8-11, the anterior portion 14 preferably includes an upper anterior segment 90 and a lower anterior segment 92 that are adjustably attachable to one another to facilitate fitting of the posterior portion 14 to a user. The upper anterior segment 90 includes a pair of upper anterior rigid members 94 and 95 preferably fixedly secured to an upper anterior laminate body 96. The lower anterior segment 92 includes a lower anterior rigid member 98 fixedly secured to a lower anterior laminate body 100.

The upper anterior rigid members 94 and 95 are each adjustably securable to the lower anterior rigid member 98, and the upper anterior laminate body is 96 is adjustably securable to the lower anterior laminate body 100. An upper interior pad 102 and a lower interior pad 104 are preferably attached to interior surfaces of the anterior laminate bodies 96 and 100, respectively, for enhanced comfort.

The upper anterior rigid members 94 and 95 are each preferably of one-piece molded plastic construction substantially similar to the material of the rigid member 24 and substantially rigid so as to resist deflection under normal use conditions.

The rigid members 94 and 95 are mirror images of one another and generally contoured to support the chin and front neck portions of the user. For example, member 94 has a generally L-shaped configuration with a leg 106 and an extension 108 substantially perpendicular to the leg 106. The leg 106 is also angled away from the plane of the extension 108 to generally correspond to the jutting of the chin of the user outwardly of the neck. Member 95 is a mirror image of the member 95 and includes a corresponding leg 110 and extension 112.

A fastener 114 extends through the free end of the extension 108 for adjusting the position of the upper rigid member 94 relative to the lower rigid member 98, as detailed below. An aperture preferably extends through the free end of the leg 106 for receiving a plastic rivet 116 for attachment of the upper anterior laminate body 96 to the upper anterior rigid member 94. Likewise, the rigid member 95 includes fastener 118 and rivet 120.

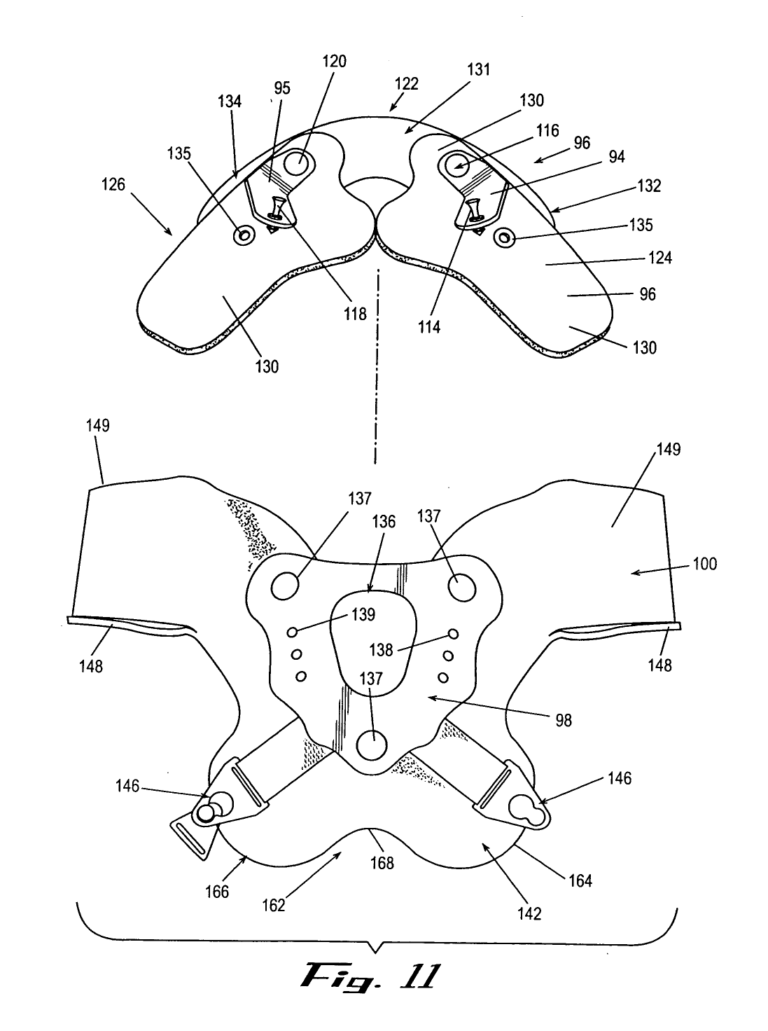

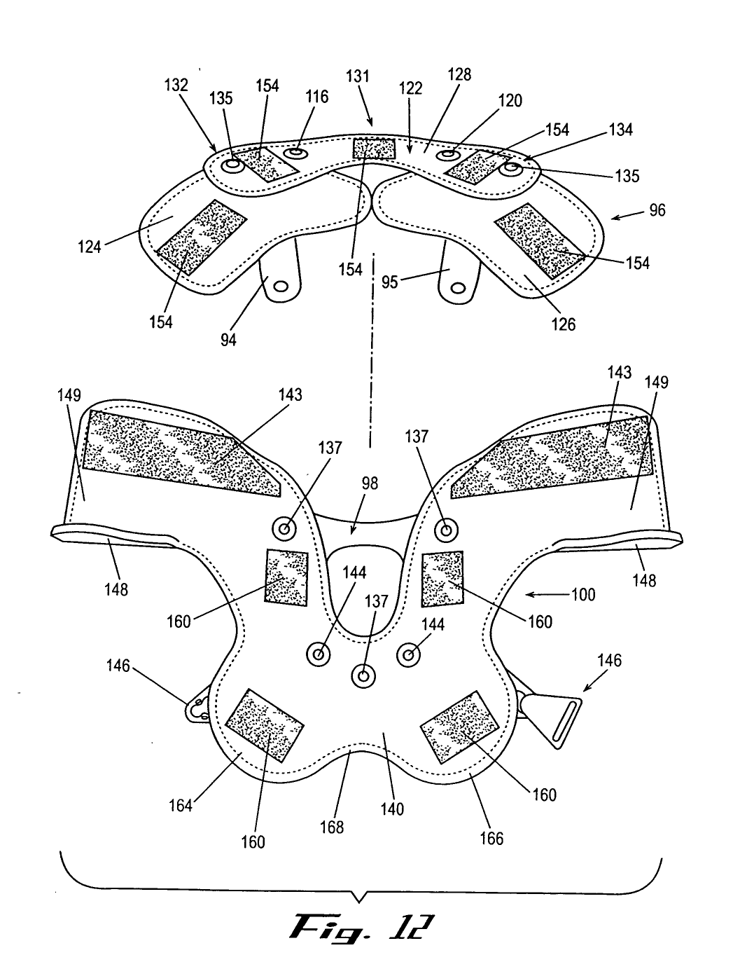

With particular reference to FIGS. 11 and 12, the upper anterior laminate body 96 is generally U-shaped and configured to generally conform to the chin and associated neck regions of the user. The body 96 is preferably provided by a central laminate member 122 connected to lateral laminate members 124 and 126.

The laminate member 122 preferably includes a low density polyethylene material 128 attached to a soft fabric material 130 having an exterior surface capable of engaging hook material in the context of mating hook and loop materials, such as VELCRO. The laminate member 122 is preferably made as by vacuum molding, press molding, and the like, with adhesive placed between the layers. The laminate members 124 and 126 are preferably made of the same laminate material as the central laminate member 122.

The central laminate member 126 has a central bight portion 131 configured to supportably receive the chin of a user, with ends 132 and 134 opposite the bight portion 131. The lateral laminate members 124 and 126 are connected to the ends 132 and 134, respectively, as by the rivets 116, 120, and preferably additional rivets 135.

The lower anterior rigid member 98 is preferably made of a rigid plastic material having a generally triangular shape and including an opening 136 located so as to be adjacent a trachea portion of the neck of the user when the collar is installed on the user. The rigid member 98 is preferably made of a plastic material similar to that used for the rigid member 24. The lower anterior rigid member 98 is preferably attached to the lower anterior laminate body 100 as by plastic rivets 137

A plurality of apertures 138 and 139 extend through the member 98 for engaging the fasteners 114 and 118, respectively, of the rigid member 98 for adjusting the position of the upper rigid member 96 relative to the lower rigid member 98.

The lower anterior laminate body 100 is generally U-shaped with the upper portion of the U extending outwardly and the bottom of the U having a bifoliar configuration. The laminate structure of the body 100 is preferably substantially similar in construction to the laminate members of the body 96, and includes a low density polyethylene material 140 attached to a soft fabric material 142 having an exterior surface capable of engaging hook material in the context of mating hook and loop materials, such as VELCRO.

Strips 143 of hook material are preferably secured to portions of the polyethylene material 140 of the lower laminate body 100 for releasably engaging the loop material of the soft fabric material 130 of the upper laminate body 96 for enabling the laminate bodies 96 and 100 to be adjustably positionable and securable to one another. Apertures preferably extend through the lower anterior laminate body for receiving plastic rivets 144 for attachment of anterior buckle assemblies 146 associated with the strap systems 18. Alternatively, the buckle assemblies 146 may preferably be attachable to and removable from the lower laminate body 100 as by use of mating hook and loop material.

To facilitate comfortable positioning of the lower laminate body 100 on the user, the lower laminate body includes ledges 148 located on outward extending arms 149 of the upper portions of the U-shape of the body 100 and configured for contacting the shoulders of the user. The ledges 148 are preferably formed during manufacture of the body 100 as by bending a portion of the body 100 in the presence of heat to impart a bend that yields the ledges 148.

Returning to FIGS. 8-10, the upper interior pad 102 is preferably made of a soft expanded foam material and includes a soft patient contact surface 150 and an opposite attachment surface 152 capable of engaging hook material in the context of mating hook and loop materials, such as VELCRO. The surface 152 of the pad 102 is releasably engageable with strips of hook material 154 adhesively secured to the laminate materials 128 of the upper laminate body 96 (FIG. 12).

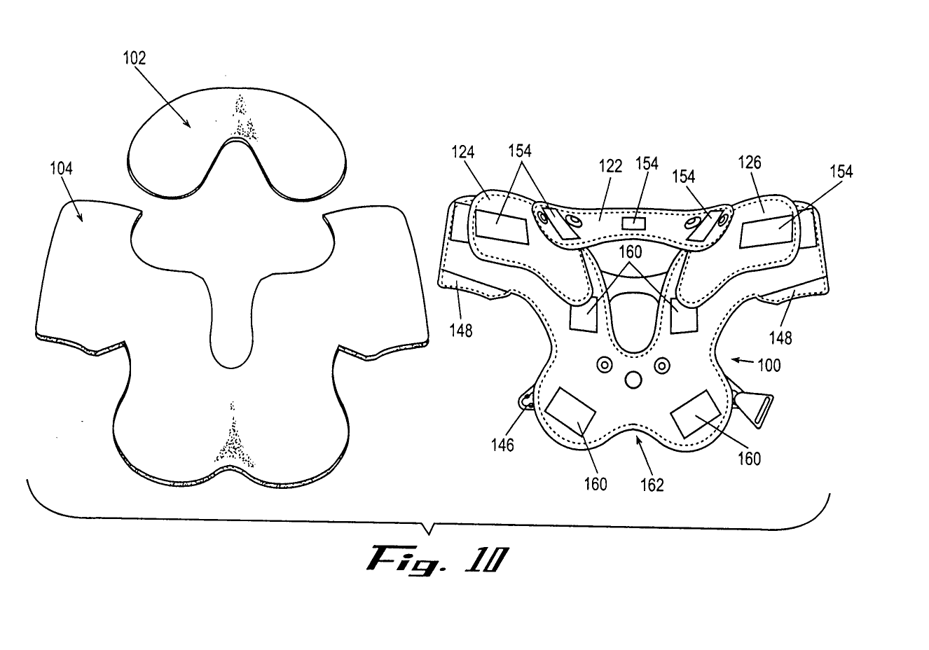

The lower interior pad 104 is preferably made of a soft expanded foam material and includes a soft patient contact surface 156 and an opposite attachment surface 158 capable of engaging hook material in the context of mating hook and loop materials, such as VELCRO. The surface 158 of the pad 104 is releasably engageable with strips of hook material 160 adhesively secured to the polyethylene material 140 of the lower laminate body 100 (FIG. 10). Alternatively, the material 140 may be finished with a loop material to engage hook material associated with the pad 104 for releasably engaging the pad 104 to the lower laminate body 100.

As noted above, the lower anterior laminate body 100 is generally U-shaped, with the upper portion of the U having the outwardly extending arms 149 and the bottom of the U having a bifoliar configuration. The arms 149 are generally configured for positioning on opposite sides of the neck of the user so that the ledges 148 are adjacent the collarbone/shoulder regions of the user.

The bifoliar configuration of the bottom portion of the laminate body 100 has been observed to facilitate fitting of the collar 10 to users and to advantageously conform to the chest area below the neck of the user and offer enhanced comfort. For example, and with particular reference to FIGS. 11 and 12, lower end 162 of the body 100 has a pair of leaves 164 and 166 which are connected by an upwardly curved segment 168. In use, the leaves 164 and 166 bear against the chest of the user, with the curved segment 168 flexing to enable the lower end 162 to substantially conform to the user.

The collar 10 may be installed on the neck of a user as by placing the posterior portion 12 against the back of the neck of the user and the anterior portion 14 adjacent the front of the user so that the chin of the user is adjacent the bight 131 and the leaves 164 and 166 bearing against the chest of the user. The ends of the anterior portion 14 preferably overlap the ends of the posterior portion 12 such that the straps 16 are adjacent the loop material of the soft fabric material 142 of the lower anterior laminate body 100. This enables the straps 16 to be readily and adjustably engaged with the material 142 to secure the collar in place and to provide a desired fit.

The flexibility of the laminate bodies 26, 30, 100, and 96 in their untensioned states advantageously enables them to substantially conform to the user for enhanced comfort and fit. The straps 16 may be adjusted to provide sufficient tension to the thus oriented and overlapped laminate bodies to render them sufficiently rigid so that the fitted and installed collar is suitable to provide the desired support.

The strap systems 18 may be utilized in conjunction with the buckle assemblies 64 and 146 to provide additional securement of the collar 10. In this regard, and with reference to FIG. 2, the strap systems 18 each preferably include an elongate padded member 170 covered with a material having an outer surface capable of engaging hook material in the context of mating hook and loop materials, such as VELCRO.

An elongate strap 172 having opposite ends 174 and 176 is attached to one of the surfaces of the padded member 170 as by stitches. The strap 172 preferably has at least some of its exposed surfaces made of a loop material. A strip of hook material 178 is attached, as by stitches, to overly strap 172 adjacent the padded member 170, and strips of hook material 180 and 182 are attached, as by stitches, to the ends 174 and 176. The strap 172 cooperates with the buckle assemblies 64 and 146 and is looped through slots of the buckle assemblies 64 ad 146 and folded back and releasably secured to hook material 178 (FIG. 1) for offering additional securement of the collar 10.

The fit of the collar 10 may be further adjusted as by loosening the straps and adjusting the relative positions of the upper and lower posterior rigid members 24 and 28, the relative positions of the upper and lower posterior laminate bodies 26 and 30, the upper and lower anterior rigid members 94, 95, and 98, and/or the upper and lower anterior laminate bodies 96 and 100. The straps 16 may then be retensioned to substantially fix the components in place and to provide sufficient rigidity to maintain the neck/chin of the user with a desired degree of support. That is, the greater the tension imparted by the straps, the more support and/or restraint is provided.

It will also be appreciated that the rigid members 24 and 28, and 94, 95, and 98 may be adjusted within the confines of their respective apertures and fasteners, the locations of which may be selected to provide a number of desired adjustment ranges. The laminate bodies 26, 30, 96, and 100 may be adjusted virtually infinitesimally and linearly and angularly by virtue of the adjustments that are enabled by the mating hook and loop material at their interfaces.

As seen in FIG. 12, the strips 143 of hook material are preferably secured to portions of the polyethylene material 140 of the lower laminate body 100 for releasably engaging the loop material of the soft fabric material 130 (FIG. 11) of the upper laminate body 96 for enabling the laminate bodies 96 and 100 to be adjustably positionable and securable to one another within a range of positions, the extremes of which are controlled by the connection between the rigid members 94, 95, and 98. In this regard, it is noted that the connections between the rigid members allow some degree of swivel or movement. Thus, within these confines the relative positions of the laminate bodies 96 and 100 may be adjusted to enable substantially custom fitting of the collar to a user.

In a similar manner, the strip of hook material 55 attached to the surface 52 of the upper laminate body 26 is matingly engageable with the loop material of the lower laminate body 30 to facilitate adjustable positioning of the upper laminate body 26 relative to the lower laminate body 30, within the confines permitted by the connections between the rigid members 24 and 28.

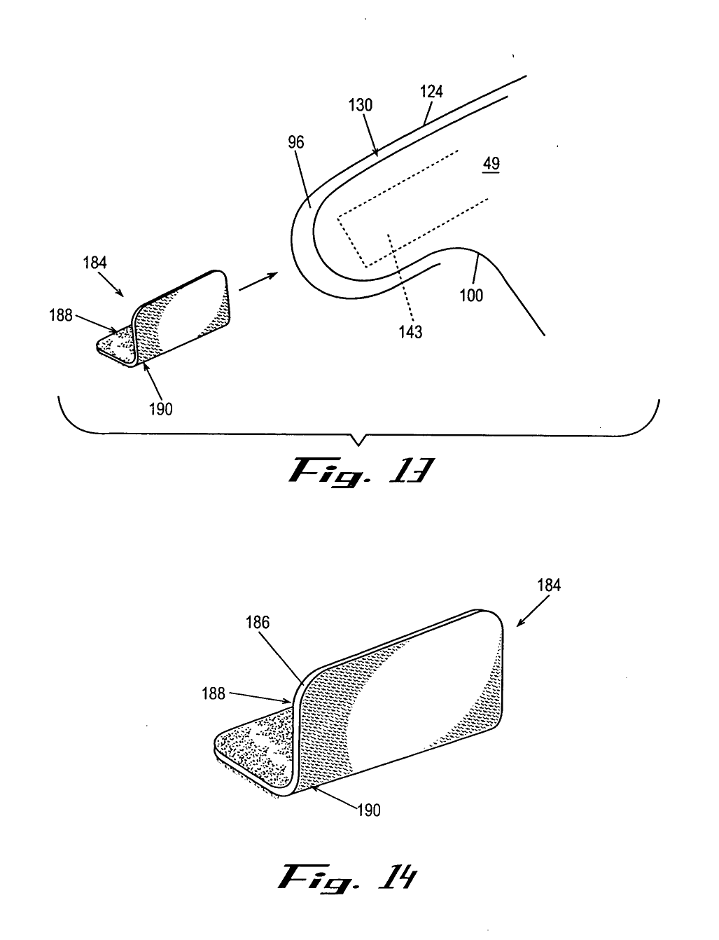

With reference to FIGS. 13 and 14, and in accordance with an alternate embodiment, the ledges 148 are replaced with ledges 184 which are adjustably positionable relative to the arms 149 of the lower laminate body 100. The ledges 184 preferably are made of a polyethylene sheet material 186 sandwiched between an outer fabric 188 and an inner fabric 190, with the resulting laminate heat molded to the desired L-shape. The outer fabric 188 preferably has a loop surface of the type suitable for engaging hook material, as in mating hook and loop material, and the inner fabric 190 preferably has a hook surface suitable for engaging loop material.

Thus, the ledges 184 may be adjustably positioned between the upper laminate body 96 and the lower laminate body 100, with the inner hook fabric 190 engaging the loop fabric material 130 of the laminate members 124 and 126 of the body 96, and the outer loop fabric 188 engaging the hook material of the strips 143 of the arms 49 of the lower laminate body 100.

With reference to FIG. 15, there is shown an alternate embodiment of the invention utilizing a supplemental strap system 192 in place of the strap system 18, and anterior buckle assemblies 194 in place of the anterior buckle assemblies 146. Each strap system 192 preferably includes an elongate strap 196 having opposite surfaces made of a hook receptive fabric. A mechanical buckle fastener, such as a post 198 for engaging a slot, is preferably attached, as by sewing, to one end of the strap 196 for releasably connecting to the buckle assemblies 64 of the posterior portion 12. A strip 200 of a loop material formed in a generally Y-shaped configuration is secured to the opposite end of the strap 196 as by pressing legs 201 and 202 of the Y-shaped strip against the surfaces of the strap 196. Extension 204 of the strip 200 may be secured to one of the surfaces of the strap 196 by the hook material thereof mating with the loop material of the strap. The extension 204 enables the length of the strap 196 to be readily trimmed for custom fitting.

The buckle assemblies 194 each preferably include a length of hook material 206 attached, as by sewing, to an extension member 208, a male buckle member 210 secured, as by sewing, to the extension member 208, and a female buckle member 212 releasably engagable with the male buckle member 210. The hook material 206 is pressed against the loop/soft fabric material 142 of the laminate body 100. The extension member 208 is preferably a sheet of polyethylene sandwich between fabric/padding layers.

To install the buckle assembly 194, the post 198 is engaged with the buckle assembly 64 and the hook material 206 attached to the fabric material 142 of the body 100. The extension 204/strap 196 may then be threaded through and looped around a slot associated with the buckle member 210, with the hook material of the extension 204 secured against the loop material of the strap 196 to complete installation.

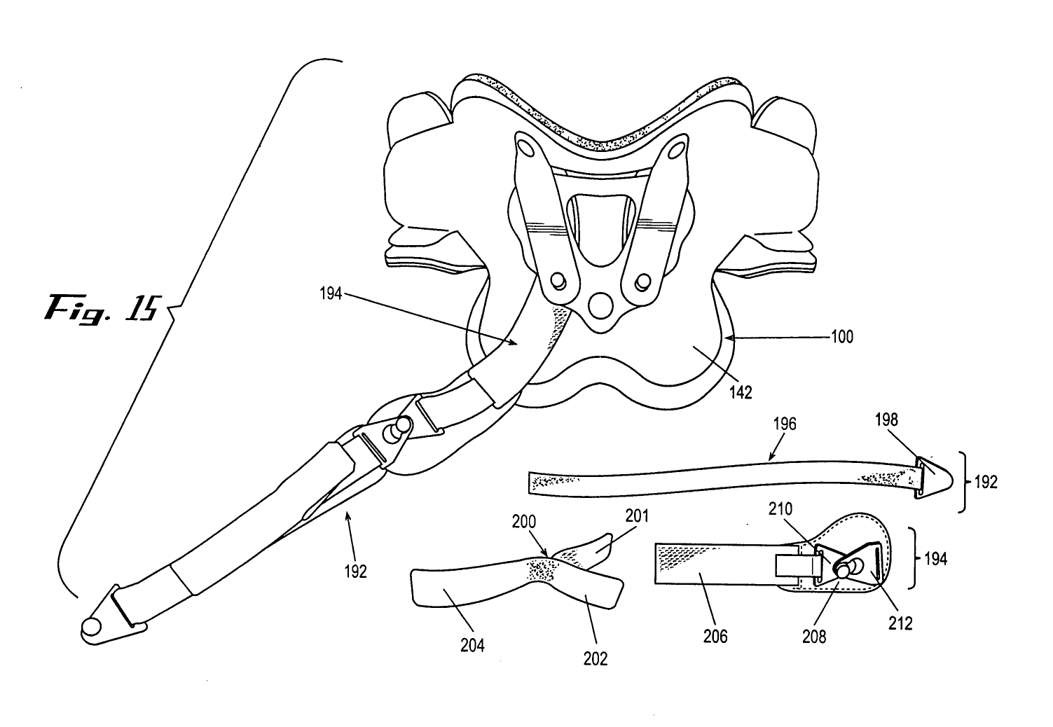

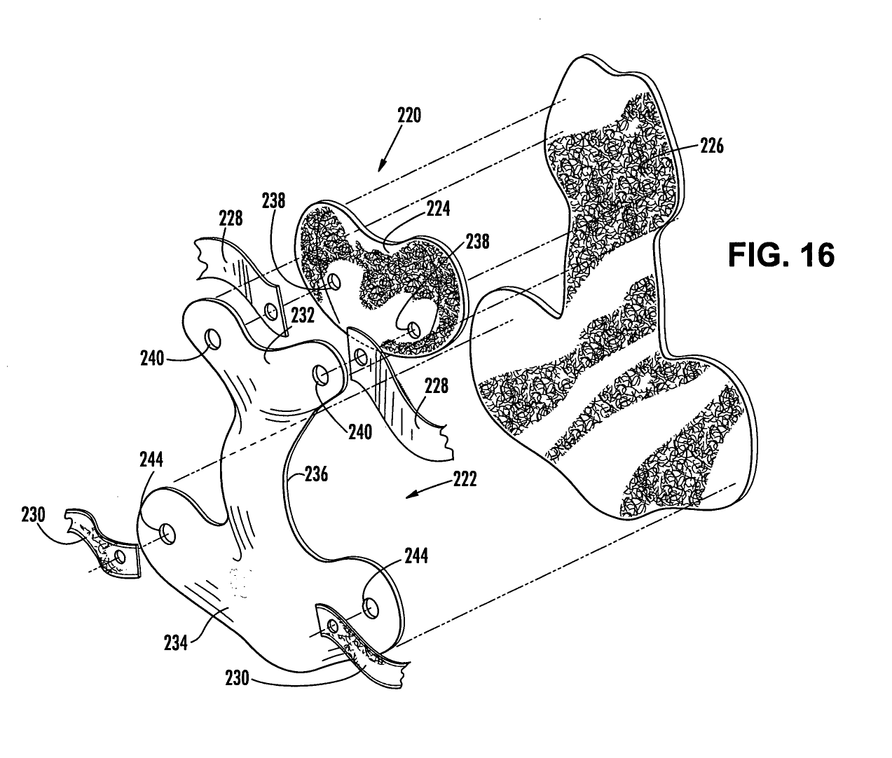

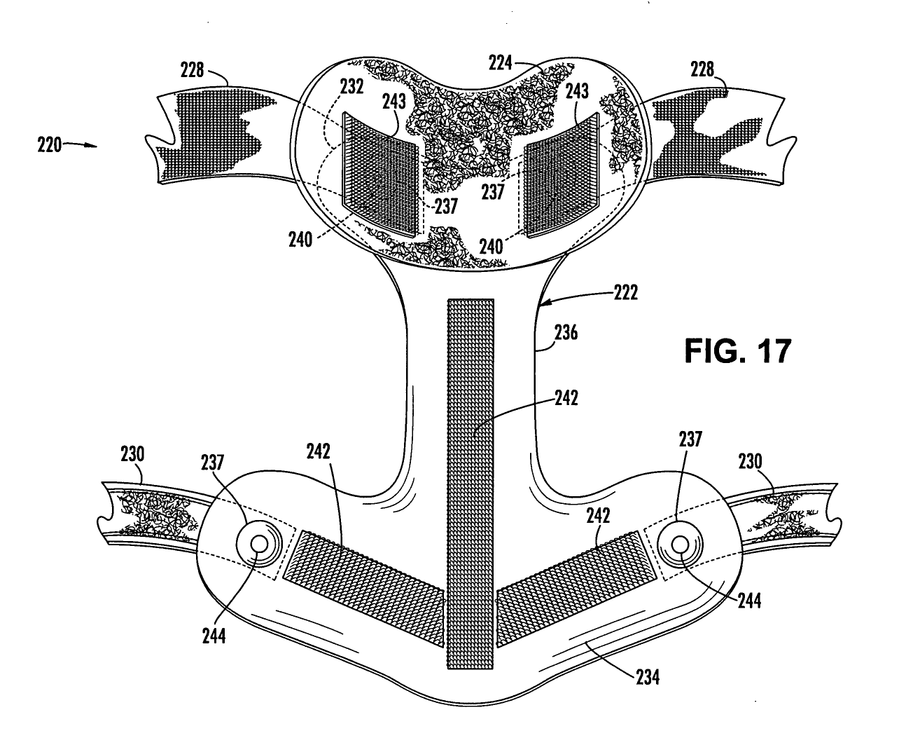

With reference now to FIGS. 16 and 17, there is shown a posterior portion 220 in accordance with an alternate embodiment of the invention. The posterior portion 220 may be used in conjunction with the anterior portion 12 described previously. In a preferred embodiment, the posterior portion 220 preferably includes a rigid member 222, a laminate member 224, a pad 226, upper strap members 228, and lower strap members 230.

The rigid member 222 is preferably of one-piece molded plastic construction and is substantially rigid so as to resist deflection under normal use conditions. The rigid member 222 may be made of the same plastic materials as the rigid members 24 and 28 and have a similar thickness.

The rigid member 222 is generally contoured to receive portions of the neck and spine of the user, and includes an upper end 232 configured to cradle the neck of the user just below the occipital protuberance of the user, a lower end 234 generally contoured to receive lower portions of the neck and spine of the user, and a connector 236 spanning between the upper end 232 and the lower end 234. The rigid member 222 is configured to correspond to the shape of the assembly provided by the rigid members 24 and 28 described above, but in a one piece construction.

The laminate member 224 is slightly larger than the upper end 232 of the rigid member 222 and generally shaped to correspond to the shape of the upper end 232. The laminate body 224 is preferably made of the same laminate material as the laminate body 26 described above and includes a foam material and a polymeric sheet material, preferably a low density polyethylene material, sandwiched between fabric materials which are preferably able to matingly engage hook material. The member 224 may be attached as by hook material adhesively to rigid member 222, and may further secured as by fasteners, such as rivets 237, which extend through apertures 238 of the member 224 and corresponding apertures 240 through the rigid member 222.

The pad 226 is slightly larger than the rigid member 222 and generally shaped to correspond to the shape thereof. The pad 226 may preferably be made of the same material as the pads 32 and 34. The pad 226 may be attached in the manner of the pads 32 and 34, such as by hook material 242 adhesively secured to the member 222 and double-sided hook material portions 242 which matingly engage the laminate material

The strap members 228 and 230 may preferably correspond to the and may be secured as by fasteners, such as the rivets 237, secured via the apertures 240 and apertures 244 through the rigid member 222. The strap members 228 and 230 may correspond to the previously described strap systems herein, such as the straps 16 and strap system 18, as well as other straps having fastening structure such as hook material or the like so as to enable the posterior portion 220 to be used in conjunction with an anterior portion similar to the anterior portion 14 and secured around the neck of a user. The straps would preferably extend over the shoulder of the user and attach to the anterior portion.

The foregoing description of certain exemplary embodiments of the present invention has been provided for purposes of illustration only, and it is understood that numerous modifications or alterations may be made in and to the illustrated embodiments without departing from the spirit and scope of the invention as defined in the following claims.

1. A cervical collar, the collar comprising:
a posterior portion generally configured to conform to a rear neck portion of a patient, the posterior portion comprising an upper posterior segment having an upper posterior rigid member connected to an upper posterior laminate body and a lower posterior segment comprising a lower posterior rigid member connected to a lower posterior laminate body, wherein the upper posterior rigid member is adjustably securable to the lower posterior rigid member and the upper posterior laminate body is adjustably securable to the lower posterior laminate body, with the upper and lower posterior laminate bodies each comprising a laminated structure having a polymeric sheet material, a foam material, and a fabric material; and
an anterior portion generally configured to conform to a chin and front neck portion of the patient, the anterior portion comprising an upper anterior segment having an upper anterior rigid member connected to an upper anterior laminate body and a lower anterior segment comprising a lower anterior rigid member connected to a lower anterior laminate body, wherein the upper anterior rigid member is adjustably securable to the lower anterior rigid member and the upper anterior laminate body is adjustably securable to the lower anterior laminate body, with the upper and lower anterior laminate bodies each comprising a laminated structure having a polymeric sheet material and a fabric material.

2. The collar of claim 1, wherein the upper and lower posterior laminate bodies each comprise laminate bodies having a foam material and a low density polyethylene material sandwiched between a pair of fabric materials and made by vacuum molding with adhesive between each layer.

3. The collar of claim 1, wherein the upper and lower anterior laminate bodies each comprise a low density polyethylene material attached to a soft fabric material.

4. The collar of claim 1, wherein the upper and lower posterior rigid members each comprise substantially rigid plastic members.

5. The collar of claim 1, wherein the upper and lower anterior rigid members each comprise substantially rigid plastic members.

6. The collar of claim 1, wherein the upper posterior rigid member includes a plurality of fasteners and the lower posterior rigid member includes a plurality of apertures, wherein the fasteners are releasably engageable with the apertures for adjusting the relative positions of the upper and lower rigid members relative to one another.

7. The collar of claim 1, wherein the upper anterior rigid member includes a plurality of fasteners and the lower anterior rigid member includes a plurality of apertures, wherein the fasteners are releasably engageable with the apertures for adjusting the relative positions of the upper and lower rigid members relative to one another.

8. The collar of claim 1, wherein the upper posterior laminate body and the lower posterior laminate body have matingly positionable surfaces that are releasably attachable to one another for permitting the laminate bodies to be adjustably positionable relative to one another.

9. The collar of claim 1, wherein the upper anterior laminate body and the lower anterior laminate body have matingly positionable surfaces that are releasably attachable to one another for permitting the laminate bodies to be adjustably positionable relative to one another.

10. The collar of claim 1, wherein he lower anterior laminate body is generally U-shaped, with the upper portion of the U having the outwardly extending arms and the bottom of the U having a bifoliar configuration

11. The collar of claim 1, further comprising straps for maintaining the anterior and the posterior portions in an overlapped orientation when installed on a user.

12. The collar of claim 12, further comprising a supplemental strap system configured to extend between portions of the posterior portion and the anterior portion and to extend adjacent an under-arm region of a user.

13. A cervical collar having upper and lower posterior laminate bodies each including a polymeric sheet material, a foam material, and a fabric material; and upper and lower anterior laminate bodies each including a polymeric sheet material and a fabric material.

14. The collar of claim 13, wherein the polymeric sheet material comprises a low density polyethylene sheet material.

15. A medical support comprising a rigid member and a laminate body securable to the rigid member, the laminate body including a polymeric sheet material, a foam material, and a fabric material.

16. The support of claim 15, wherein the polymeric sheet material comprises a low density polyethylene sheet material.

17. The support of claim 15, wherein the medical support further comprises a strap and wherein the laminate body is substantially flexible in an untensioned state so as to be positionable about a portion of a user, yet which becomes sufficiently rigid when positioned about the portion of a user and tensioned as by the use of the strap.

18. The support of claim 15, wherein the support is a cervical collar.
